(19) United States
(12) Patent Application Publication
(10) Pub. No.: US 2022/0339017 A1
(43) Pub. Date: Oct. 27, 2022

(54) SYSTEM AND METHOD FOR ADDITIVELY MANUFACTURING AN ANKLE FOOT ORTHOSIS

(71) Applicant: THE REGENTS OF THE UNIVERSITY OF MICHIGAN, Ann Arbor, MI (US)

(72) Inventors: Albert J. Shih, Ann Arbor, MI (US); Robert Chisena, Ann Arbor, MI (US); Ryan Posh, Ann Arbor, MI (US); Miguel Funes, Ann Arbor, MI (US); Darren Bolger, Ann Arbor, MI (US); Jeff Wensman, Ann Arbor, MI (US)

(21) Appl. No.: 17/761,914

(22) PCT Filed: Sep. 17, 2020
(86) PCT No.: PCT/US20/51164

§ 371 Date: Mar. 18, 2022

Related U.S. Application Data
(60) Provisional application No. 62/902,685, filed on Sep. 19, 2019.

(51) Int. Cl. A61F 5/01; B33Y 80/00; B33Y 10/00; B33Y 30/00; B33Y 50/02; B29C 64/393

(52) U.S. Cl. CPC A61F 5/0111; B33Y 80/00; B33Y 10/00; B33Y 30/00; B33Y 50/02; B29C 64/393

(57) ABSTRACT

An additively manufactured ankle-foot orthosis. The ankle-foot orthosis includes an additively manufactured footplate and a calf cuff separate from the additively manufactured footplate. A pre-fabricated strut connects the additively manufactured footplate to the additively manufactured calf cuff and includes thickness and width adapted to define a patient-specific stiffness about an ankle joint of a patient. The stiffness of the pre-fabricated strut is suited to one or more of a gait need or a gait requirement of each patient, and the additively manufactured footplate has a portion with a shape complementary to a shape of a portion of the pre-fabricated strut. So configured, a collective shape and volume of the additively manufactured footplate and the pre-fabricated strut together are adapted to fit inside a shoe of the patient.

### CROSS REFERENCE TO RELATED APPLICATIONS

This application claims the benefit of U.S. Provisional Application No. 62/902,685 filed Sep. 19, 2019. The entire content of this application is incorporated herein by reference in its entirety.

### STATEMENT OF FEDERALLY SPONSORED RESEARCH AND DEVELOPMENT

This invention was made with government support under Grant Number IIP1534003 awarded by the National Science Foundation. The government has certain rights in the invention.

### FIELD OF THE DISCLOSURE

This disclosure relates generally to ankle-foot orthoses (AFOs) and, more specifically, a system and method of additively manufacturing the AFO without using chemicals to dissolve support material.

### BACKGROUND OF THE DISCLOSURE

Currently, ankle-foot orthoses, as shown in FIGS. 1A-1C, are orthotic devices designed to support and offer stability to the ankle and foot, constrain the motion of the ankle joint, and compensate for weaknesses. AFO is the second most commonly used orthosis in the United States today, accounting for 26% of all orthotic devices. AFOs are used by patients with neuromuscular disorders, such as myotonic dystrophy and stroke patients with drop-foot syndrome. AFOs are also common for diabetic patients who experience numbness and loss of control over their foot and ankle, a condition known as neuropathy.

To meet the needs of a wide range of patients, different AFO designs exist. Generally, the most prevalent designs are the pre-fabricated (pre-fab) AFO, the traditional custom AFO, and the three-part custom AFO.

A pre-fab AFO, also known as an off-the-shelf AFO is a non-customized orthosis AFO 2 that is manufactured in mass-production. An example of the pre-fab AFO 2 by SPS is shown in FIG. 1(a). Pre-fab AFOs are designed using a generic leg template with different sizes. When a patient requires an AFO, the “best-fit” size is chosen and can be modified by locally heating to deform and conform to the patient's shape. However, without full customization, pre-fab AFOs may lead to localized discomfort, skin irritation, or the development of open sores. For patients with neuropathy, edema, easy bruising of sensitive skin, or other pathologies, custom AFOs are required.

Custom AFOs are designed and fabricated to meet an individual's shape and needs. There are two types of custom AFOs: a traditional one-piece custom AFO 4 and a three-part custom carbon fiber composite AFO 6. FIG. 1(b) shows the one-piece custom AFO 4 made of polypropylene (PP) or PP/polyethylene (PE) co-polymer. This type of one-piece AFO is fabricated by first creating and modifying a plaster replica of the patient's foot and leg geometry in a labor-intensive and time-consuming task. FIG. 1(c) shows the three-part AFO 6 originally invented to accommodate more active users. These three-part AFOs, such as the ExoSym™ Leg Brace (formerly the Intrepid Dynamic Exoskeletal Orthosis or IDEO) or the Posterior Dynamic Element (PDE™), are composed of three key components: a footplate 6a, a calf support 6b, and a strut 6c. The footplate 6a and calf support 6b have the shell structure and are custom made using lightweight carbon fiber composite material. The footplate 6a and calf support 6b are connected by a strut 6c, such as a thin strut, which is also made from carbon fiber composite material with a specific bending stiffness. This advanced, lightweight AFO is designed for active users. This type of three-part custom carbon fiber AFO costs more and takes a longer time to fabricate in comparison to the traditional one-piece custom AFO due to the layup and curing of carbon fiber custom parts. Custom AFOs have better efficacy in clinical trials because their shapes match and conform to a patient's foot and leg for comfort and fit.

In addition, the foregoing custom AFOs typically do not fit within a shoe of the patient, preventing the patient from wearing a normal, comfortable shoe while wearing the custom AFO. For example, some AFOs do not include collective shapes for different parts that might enable such parts to fit within the shoe of the patient.

Further, the load from walking when the patient is using the AFO is not uniformly spread to the ankle region of the footplate, for example. As a result, the load is often not below the combined fatigue and impact failure strength of the material of the footplate, resulting in an increased risk for damage, bending, and wear to the footplate of such conventional AFOs.

AFOs utilize the gait dynamics of the individual and act as a spring to store and release energy to the patient's lower limb. Much like a dynamic ankle and foot prosthesis, the AFO stores energy when deformed in midstance and then releases that energy at the end of stance. The dynamics depend on the stiffness and damping of the strut and footplate and is important and unique to each individual. The AFO stiffness also determines the ankle's range of motion, the amount of energy return while walking, and the extent to which the ankle remains in a neutral position. For the three-part AFOs, as the patient progresses through their rehabilitation, the strut can be changed for different dynamic responses. The 3-part AFO design has demonstrated positive outcomes.

The current practice to manufacture the custom AFO is based on the technique of conformable plaster molding of the patient's body. FIG. 2 shows the five steps that are employed to fabricate the AFO: 1) casting a negative impression of the foot and leg using plaster tape, as in block 8a; 2) creating a positive plaster mold using the plaster tape in Step 1 as the mold, as in block 8b; 3) modifying the plaster mold in Step 2 by removing and adding plaster in high and low contact pressure regions, respectively, as in block 8c; 4) vacuum forming a semi-molten PP or PP/PE copolymer plastic sheet around the modified plaster mold, as in block 8d; and 5) trimming and finishing the AFO to the final shape, as in block 8e.

Much like their one-piece thermoplastic counterpart, three-part AFOs are manufactured by first creating and modifying a plaster replica of the patient's foot and leg, as described in FIG. 2. Utilizing this plaster mold as the patient's leg geometry, calf, and footplate, sections are made from carbon fiber composite layups. A technician stretches the carbon fiber lay-ups across the model manually layer-by-layer until the desired thickness is obtained. Once the carbon fiber is cured, the trim-line of the AFO is determined. Finally, the appropriate strut is then selected for the patient, or in some cases also customized and manufactured.

Both the traditional thermoplastic and carbon fiber composite layering methods are labor-intensive, time-consuming, costly, and experience-based. The traditional custom AFO can take between 1 to 3 weeks to manufacture. The carbon fiber composite three-part AFOs require even longer time, due to the use of carbon fiber layups as well as the design complexity. A proper AFO utilizing these processes requires highly trained orthoptists, skilled technicians, and many patient visits, which is time consuming and costly.

This current design and manufacturing practice for custom AFOs as well as other custom assistive devices are rudimentary and utilize primitive tools. The current approach has four major drawbacks: (1) Time-consuming and long delivery time (e.g. AFOs typically need one week turnaround time); (2) Artisan-based manufacturing wherein inaccuracy depends mainly on the skills of the clinician/technician, and the rudimentary techniques to create molds; (3) Multiple visits to the clinic for adjustment and fit are required; and (4) No digital patient records are kept to track the progress of the shape or the patients' symptoms, which provide a measure of the efficacy of treatment.

In addition to custom AFOs, there are many types of custom orthoses, prostheses, and assistive devices, including the foot orthosis (FO), thoracic lumbar sacral orthosis (TLSO), above-knee (AK) and below-knee (BK) prosthesis, cervical orthosis (CO), knee AFO (KAFO), lumbosacral orthosis (LSO), and others. Most of these custom assistive devices are made by the plaster and carved foam block molding approach, such as the approach depicted in FIG. 2.

### SUMMARY OF THE DISCLOSURE

According to one aspect of the present disclosure, an ankle-foot orthosis comprises an additively manufactured footplate and a calf cuff separate from the additively manufactured footplate. A pre-fabricated strut connects the additively manufactured footplate to the calf cuff and includes a thickness and width adapted to define a patient-specific stiffness about an ankle joint of a patient. The pre-fabricated strut also includes a portion with a shape, such as a curvature.

According to another aspect of the present disclosure, an ankle-foot orthosis comprises an additively manufactured footplate and a calf cuff separate from the additively manufactured footplate. A pre-fabricated strut connects the additively manufactured footplate to the calf cuff and includes a thickness and width adapted to define a patient-specific stiffness about an ankle joint of a patient. The stiffness of the pre-fabricated strut is suited to one or more gait needs or requirements of each patient.

According to yet another aspect of the present disclosure, an ankle-foot orthosis comprises an additively manufactured footplate and a calf cuff separate from the additively manufactured footplate. A pre-fabricated strut connects the additively manufactured footplate to the calf cuff and includes a thickness and width adapted to define a patient-specific stiffness about an ankle joint of a patient. The pre-fabricated strut also includes a portion with a shape. The additively manufactured footplate has a portion with a shape complementary to the shape of the portion of the pre-fabricated strut.

According to yet another aspect of the present disclosure, an ankle-foot orthosis comprises an additively manufactured footplate and a calf cuff separate from the additively manufactured footplate. A pre-fabricated strut connects the additively manufactured footplate to the calf cuff and includes a thickness and width adapted to define a patient-specific stiffness about an ankle joint of a patient. A collective shape and volume of the additively manufactured footplate and the pre-fabricated strut together are adapted to fit inside a shoe of the patient.

According to another aspect of the present disclosure, an ankle-foot orthosis comprises an additively manufactured footplate having an ankle region, and a calf cuff separate from the additively manufactured footplate. A pre-fabricated strut connects the additively manufactured footplate to the calf cuff and includes a distal end having an anchor with two fingers forming a two-finger expansion. The anchor has a center portion with a hole, and each finger includes a hole. In addition, the anchor is adapted to uniformly spread the load from walking to the ankle region of the additively manufactured footplate, where the load is below the combined fatigue and impact failure strength of a material of the additively manufactured footplate. For this reason, the durability of the footplate is improved. In addition, the he anchor reduces the thickness of the ankle region, allowing the shape and volume of the additively manufactured footplate to fit inside a shoe of a patient.

According to another aspect of the present disclosure, a method of additively manufacturing an ankle-foot orthosis comprises providing a tree-shape structure as a support structure for material extrusion of the ankle-foot orthosis and fabricating the tree structure support and one or more of a footplate and a calf cuff with a single material. The method further includes eliminating support material used in material extrusion and removing the tree support structure from one or more of the footplate and the calf cuff without using any chemicals for dissolving any support material. This reduces the printing time and helps ensure the safety of all patients, for example. According to yet another aspect of the present disclosure, a system for additively manufacturing an ankle-foot orthosis comprises a communication network, and a scanning device communicatively coupled to the communication network. The scanning device includes a memory and at least one processor, that executes a scanning module stored on the memory of the scanning device to create a patient-specific scan. The system further includes a 3D printer communicatively coupled to the communication network and a cyber design system having a computing device communicatively coupled to the communication network, the scanning device, and the 3D printer. The computing device of the cyber design system includes a memory, at least one processor, a transmitter, and a receiver and receives data from the scanning device relating to the patient-specific scan. Further, a module is stored in the memory of the computing device of the cyber design system and executable by the at least one processor of the computing device of the cyber design system to (1) receive data from the scanning device relating to the patient-specific scan; and (2) create a patient-specific ankle-foot orthosis profile based at least in part on the data received from the scanning device, the patient-specific ankle-foot orthosis profile adapted to be transmitted to the 3D printer to implement the patient-specific ankle-foot orthosis profile. The scanning device creates the patient-specific scan and the 3D printer receives the patient-specific ankle-foot orthosis profile from the computing device of the cyber design center. The 3D printer then implements the patient-specific ankle-foot orthosis profile to additively manufacture a footplate of an ankle-foot orthosis and a calf cuff of the ankle-foot orthosis, the calf cuff separate from the footplate, within one day.

### BRIEF DESCRIPTION OF THE SEVERAL VIEWS OF THE DRAWINGS

FIG. 1A is a conventional prefabricated ankle-foot orthosis;

FIG. 1B is a conventional custom ankle-foot orthosis;

FIG. 1C is another conventional custom ankle-foot orthosis;

FIG. 2 is a block diagram depicting a conventional design and fabrication process of a conventional custom ankle-foot orthosis;

FIG. 3A is a perspective view of a system for additively manufacturing an ankle-foot orthosis (AFO) according to one aspect of the present disclosure;

FIG. 3B is a block diagram of a portion of the system for additively manufacturing an AFO of FIG. 3A;

FIG. 4A is a perspective view of an additively manufactured ankle-foot orthosis according to an aspect of the present disclosure;

FIG. 4B is a block diagram of an inertia measurement unit of the additively manufactured ankle-foot orthosis of FIG. 4A;

FIG. 5 is a perspective view of a portion of the additively manufactured ankle-foot orthosis of FIG. 4;

FIG. 6 is a front view of another portion of the additively manufactured ankle-foot orthosis of FIG. 4;

FIG. 7 is a rear view of an additively manufactured footplate of the additively manufactured ankle-foot orthosis of FIG. 5;

FIG. 8 is a front view of the additively manufactured footplate of FIG. 7;

FIG. 9 is a perspective view of another additively manufactured ankle-foot orthosis according to another aspect of the present disclosure;

FIG. 10 is a perspective view of another additively manufactured ankle-foot orthosis according to another aspect of the present disclosure;

FIG. 11 is a perspective view of the additively manufactured footplate and part of a pre-fabricated strut of the additively manufactured ankle-foot orthosis of FIG. 4, with a patient's foot and calf disposed therein;

FIG. 12 is a perspective view of a pair of additively manufactured footplates of the present disclosure with the patient's feet disposed therein;

FIG. 13 is a top view of the additively manufactured footplate of the present disclosure with a portion of a patient's foot and ankle disposed therein;

FIG. 14 is a side perspective view of the additively manufactured footplate of the present disclosure with a portion of the patient's foot and ankle disposed therein;

FIG. 15 is a perspective view of another additively manufactured ankle-foot orthosis according to another aspect of the present disclosure;

FIG. 16 is a perspective view of the additively manufactured footplate of FIG. 4 coupled to at least one tree support structure of the additive manufacturing method of the present disclosure;

FIG. 17 is another perspective view of the additively manufactured footplate of FIG. 4 separated from the at least one tree support of FIG. 18; and

FIG. 18 is another perspective view of the additively manufactured footplate of FIG. 4 separated from the at least one tree support of FIG. 18.

### DETAILED DESCRIPTION OF THE PREFERRED EMBODIMENTS

In a preferred embodiment of the present disclosure, an ankle-foot orthosis includes an additively manufactured footplate and an additively manufactured calf cuff separate from the additively manufactured footplate. A pre-fabricated strut connects the additively manufactured footplate to the additively manufactured calf cuff and includes thickness and width adapted to define a patient-specific stiffness about an ankle joint of a patient. The stiffness of the pre-fabricated strut is suited to one or more gait needs or requirements of each patient, and the additively manufactured footplate has a shape complementary to the shape of the pre-fabricated strut. The collective shape and volume of the additively manufactured footplate and the pre-fabricated strut together are adapted to fit inside a shoe of the patient, and the additive manufacturing time is reduced to less than three hours by additively manufacturing the footplate separate from the calf cuff. This reduction in manufacturing time allows a one-day visit with the patients coming to the clinic in the morning and leaving by the end of the day wearing the evaluated and adjusted AFO. As a result, there is a significant improvement in the manufacturing turnaround time for the ankle-foot orthosis of the present disclosure compared to conventional AFOs typically taking at least one to three weeks to manufacture.

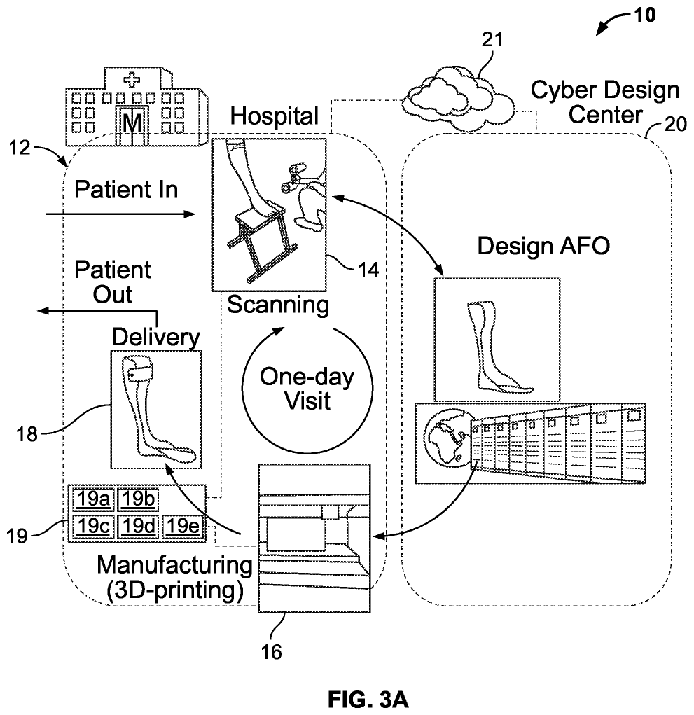

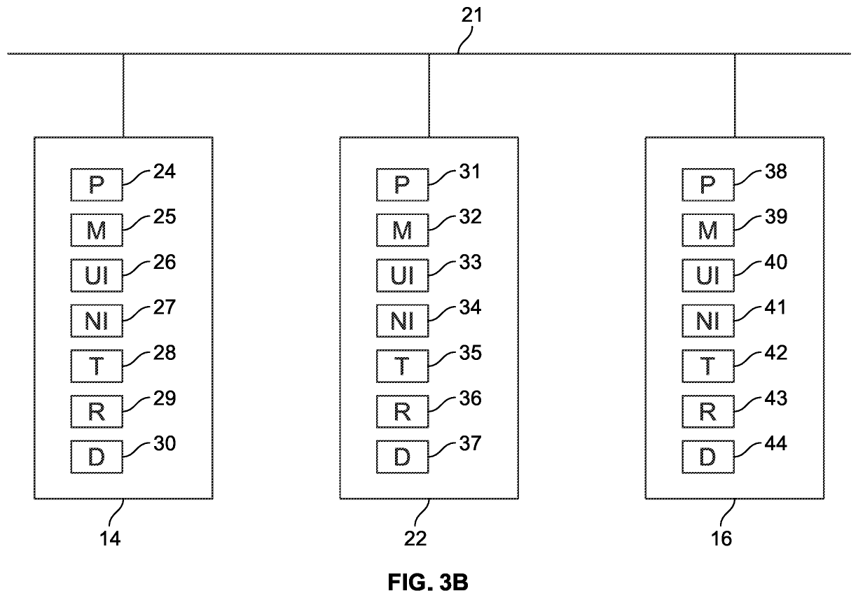

Referring now to FIG. 3A, a system 10 for additively manufacturing an ankle-foot orthosis is depicted. The system 10 includes a patient center 12, such as a hospital, clinic, or other similar patient location. The patient center 12 includes a scanning device 14 that scans a patient's body part, such as the patient's calf, ankle and/or foot, to create a patient-specific scan for the ankle-foot orthosis. The scanning device 14 may include one or more various known computing devices, such as an optical shape scanner, an iPad, an iPod, an iPhone or any other smartphone, tablet or other device having such optical non-contact scanning capabilities, for example. The patient-specific scan is later used to make the additively manufactured ankle-foot orthosis via a 3D printer, as described more below. The patient center 12 further includes a 3D printer 16, which additively manufactures the patient-specific ankle-foot orthosis, and a delivery center 18 for delivery of final and complete additively manufactured ankle-foot orthosis. A data station 19 is also communicatively coupled via one or more of a wired or wireless network to each of the scanning device 14 and the 3D printer 16. The data station 19 may also be communicatively coupled to other parts of the patient center 12 and still fall within the scope of the present disclosure. While the data station 19 is depicted as part of the patient center 12 in FIG. 3A, it will be understood that the data station 19 may alternatively be disposed outside of the patient center 12 in a location different from and/or remote from the patient center 12 and still fall within the scope of the present disclosure. The data station 19 includes at least one processor 19a, a memory 19b, a transmitter 19c, a receiver 19d, and a network interface 19e. So configured, the receiver 19d receives data, the processor 19a processes the data according to a module stored on the memory 19b, and the network interface 19e enables the data station 19 to be communicatively coupled to one or more of the scanning device 14, the 3D printer 16 or a part of an ankle-foot orthosis, as explained more below.

As further depicted in FIG. 3A, the system 10 further includes a cyber design system 20 communicatively coupled to the patient center 12. In one example, the cyber design system 20 is communicatively coupled via a communication network 21, such as wireless network 21. As depicted in FIG. 3, the cyber design system 20 includes one or more computing devices 22 that may receive data corresponding to a scan of the patient's ankle, foot, and calf via the scanning device 14 of the patient center 12. At least one computing device 22 of the cyber design system 20 then sends the patient-specific design to the 3D printer 16, such as via the wireless network 21, directing the 3D printer 16 to additively manufacture an ankle-foot orthosis according to the patient-specific design.

Referring to FIG. 3B, in one example, the scanning device 14 includes one or more processors 24 that implement a scanning module stored in a memory 25 of the scanning device 14 to scan one or more of a foot, calf, and/or ankle of the patient. The scanning device 14 may also include a user-input 26 and a network interface 27, which allows the scanning device 14 to be communicatively coupled to the wireless network 21, for example, and communicate with the cyber design center 20. The scanning device 14 further includes a transmitter 28 and a receiver 29, such that the transmitter 28 transmits scanned data corresponding to the patient-specific scan (e.g., of one or more of the foot, calf and/or ankle of the patient) to the cyber design center 20 for processing, as explained more below. Further, the scanning device 14 may also include a display 30 on which the scanned data corresponding to the patient may be displayed, for example.

Similarly, the one or more computing devices 22 of the cyber design system 20 also includes one or more processors 31 that implement a module stored in a memory, such as a memory 32 of the computing device 22, to receive and process data corresponding to the patient-specific scan from the scanning device 14. The computing device 22 may also include a user-input 33 and a network interface 34, which allows the computing device to be communicatively coupled to the wireless network 21 and communicate with both the scanning device 14 and the 3D printer 16. The cyber design system computing device 22 may also include a transmitter 35 and a receiver 36, such that the transmitter 35 transmits processed data relative to a patient-specific scan (e.g., from the scanning device 14) to the 3D printer 16, directing the 3D printer 16 to print an ankle-foot orthosis according to the patient-specific scan. The receiver 36 receives scanning data from the scanning device 14, which is processed by one or more processors 31 of the computing device 22 and used to implement the operation of the 3D printer 16. The computing device 22 also includes a display 37, on which data, such as data from the scanning device 14 and data processed by the computing device 22, may be displayed.

Still referring to FIG. 3B, the 3D printer 16 of the patient center 12 is communicatively coupled to both the scanning device 14 and the cyber design system 20. The 3D printer 16 includes one or more processors 38 that implement a patient-specific orthosis profile created and then transmitted from the computing device 22 of the cyber design system to the 3D printer 16 and stored in a memory 39 of the 3D printer 16. The patient-specific orthosis design profile that may be stored in the memory 39 of the 3D printer 16 includes a patient-specific design protocol for execution by one or several processors 38 of the 3D printer 16. The 3D printer 16 may also include a user-input 40 and a network interface 41, which also allows the 3D printer 16 to be communicatively coupled to the wireless network 21, for example. The 3D printer 16 further includes a transmitter 42, a receiver 43 for receiving data from the cyber design system 20 relative to a patient-specific orthosis profile, for example, and a display 44, which may include or be separate from the user-input 40.

Each of the processors 24, 31, and 38 may be a general processor, a digital signal processor, ASIC, field-programmable gate array, graphics processing unit, analog circuit, digital circuit, or any other known or later developed processor. The processor 24 of the scanning device 14 may operate according to a profile stored in the memory 25 of the scanning device 14, for example. The memory 25, 32, 39 may be a volatile memory or a non-volatile memory. The memory 25, 32, 39 may include one or more of read-only memory (“ROM”), random-access memory (“RAM”), a flash memory, an electronic erasable program read-only memory (“EEPROM”), or other types of memory. The memory 25, 32, 39 may include an optical, magnetic (hard drive), or any other form of data storage.

In one example, the patient-specific orthosis design protocol is part of the patient-specific design profile stored on the memory 32, 39, and includes a set of executable instructions that control the 3D printer 16 to print the patient-specific orthosis, such as the ankle-foot orthosis. The patient-specific orthosis design protocol may be stored on the memory 32, 39 as computing logic, which includes one or more routines and/or sub-routines, embodied as computer-readable instructions stored on the memory 32, 39. The processor 31, 38 can execute the logic to cause the processor 31, 38 to retrieve the profile and control the 3D printer 16 in accordance with the patient-specific orthosis design profile. In particular, the patient-specific orthosis design protocol may specify, among other parameters, the size, shape, and/or volume of each of a footplate and a calf cuff of an ankle-foot orthosis and the timing of the 3D printing.

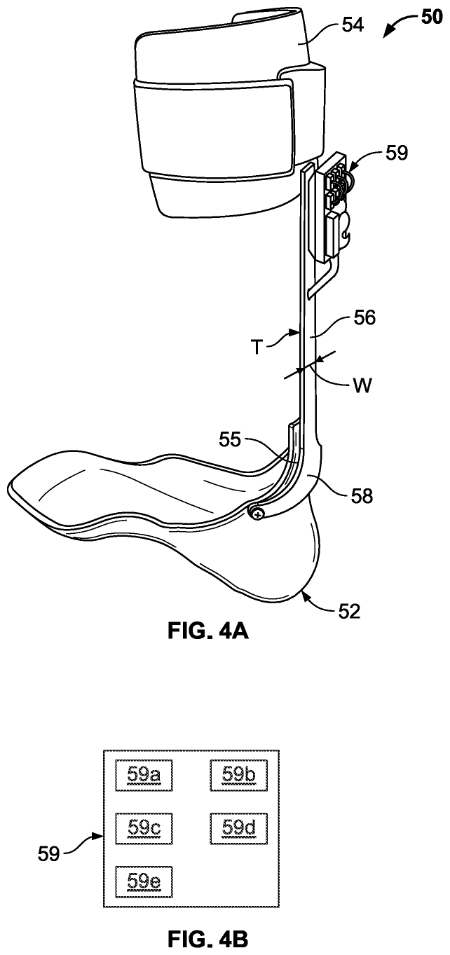

Referring now to FIG. 4A, an exemplary additively manufactured ankle-foot orthosis 50 according to an aspect of the present disclosure is depicted. The additively manufactured ankle-foot orthosis 50 is created by the system 10 described above, according to a novel additive manufacturing method described more below. The additively manufactured ankle-foot orthosis 50 includes an additively manufactured footplate 52 and a calf cuff 54 separate from the additively manufactured footplate 52. In one example, the calf cuff 54 may also be additively manufactured. The additively manufactured footplate 52 includes an ankle region 55. A pre-fabricated strut 56 connects the additively manufactured footplate 52 to the calf cuff 54 and includes a thickness T and a width W adapted to define a patient-specific stiffness about an ankle joint of a patient (not depicted), as explained more below. The pre-fabricated strut 56 may take the form of various different shapes and sizes and still fall within the scope of the present disclosure. For example, the pre-fabricated strut 56 may have a C-, V- or I-shaped cross-section to increase bending stiffness, for example. Of course, various other shapes may alternatively be used. The pre-fabricated strut 56 also includes a portion 58 with a shape, and an inertia measurement unit 59 disposed on the pre-fabricated strut 56 near the calf cuff 54. The shape of the portion 58 may take the form of various different shapes and sizes and still fall within the scope of the present disclosure. In addition, the inertia measurement unit 59 is adapted to measure one or more gait motions and patient data during the use of the ankle-foot orthosis 50, which is also explained in more detail below. The patient-specific stiffness of the pre-fabricated strut 56 is suited to one or more of a gait need or a gait requirement of each patient. In one example, the pre-fabricated strut 56 comprises metal, such as AISI spring steel. However, other materials may alternatively be used and still fall within the scope of the present disclosure.

Referring to FIG. 4B, the inertia measurement unit 59 includes a processor 59a, memory 59b, a transmitter 59c, and a receiver 59d, such that the receiver 59d receives data one or more of detected, sensed, and/or measured by the inertia measurement unit 59. The processor 59a processes the data according to at least one module stored on the memory 59b, and the transmitter 59c transmits data to the data station 19 in real-time or every specific time via the wireless network 21, for example. The data station 19 then uses the collected data to estimate one or more of a step height, a step length, and a step duration of the patient, for example. In addition, the data station 20 one or more of saves, maintains, and tracks data relative to records of the patient. Such data and records include one or more of the shape of the foot, ankle, and/or calf of the patient before and after use of the ankle-foot orthosis 50. The data also includes a range of motion of the foot, ankle, and/or calf during the process of rehabilitation. As will be appreciated, various other parameters of the patient may additionally and/or alternatively be collected and/or estimated and still fall within the scope of the present disclosure.

In some examples, the inertia measurement unit 59 includes a sensor, such as a strain gauge, and/or any other commonly used sensor having the same or similar capabilities of the aforementioned inertia measurement unit 59. In addition, while the inertia measurement unit 59 is disposed on the pre-fabricated strut 56 near the additively manufactured calf cuff 56, the inertia measurement unit 59 may alternatively be disposed on other areas of the pre-fabricated strut 56, such as near the additively manufactured footplate 52 and still fall within the scope of the present disclosure. Still further, in another example, the inertia measurement unit 59 may alternatively be disposed within a portion of the additively manufactured footplate 52, such that when the additively manufactured footplate 52 is being manufactured, a channel is created within the layer-by-layer process, and the channel is adapted to receive the inertia measurement unit 59. Such alternatives are also still within the scope of the present disclosure.

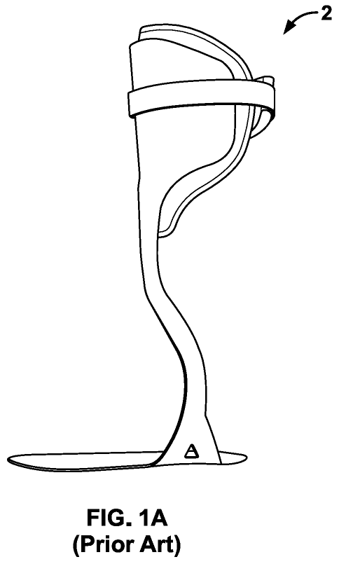

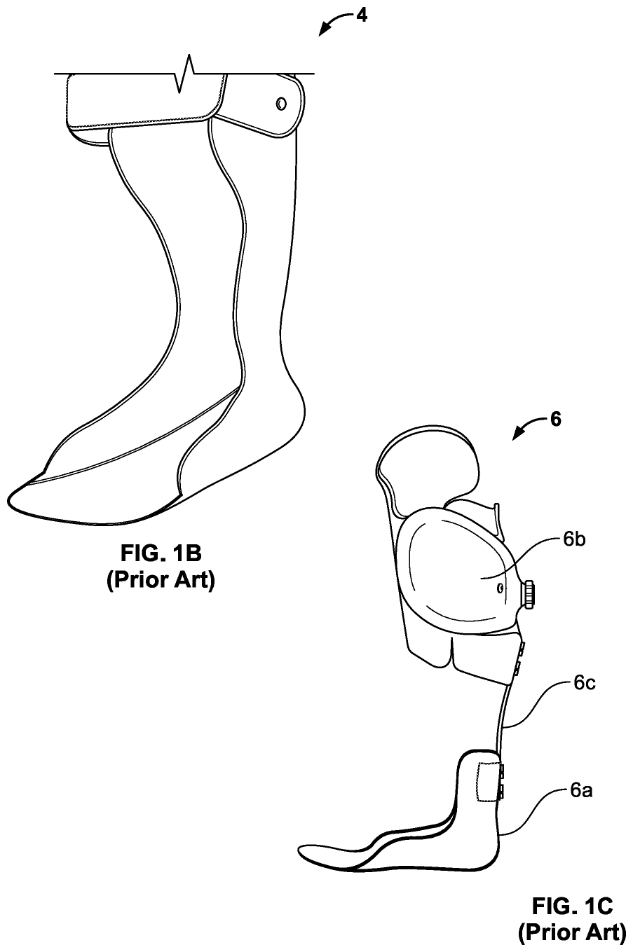

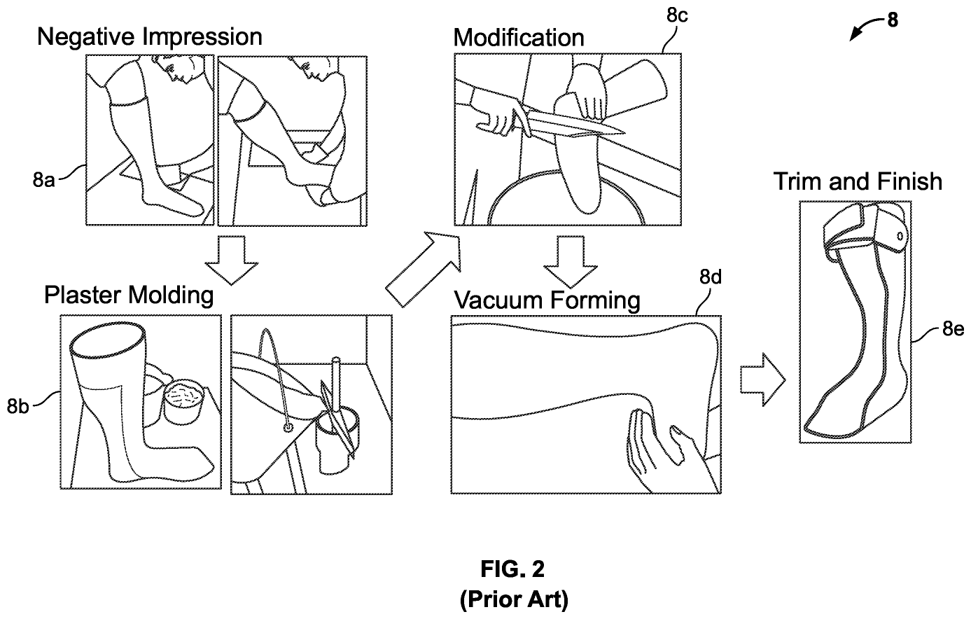

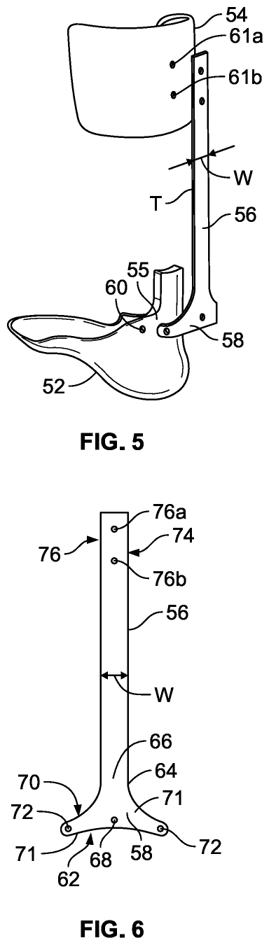

Referring now to FIG. 5, the additively manufactured footplate 52 also includes a portion 60 with a shape that matches the shape of the portion 58 of the pre-fabricated strut 56. Said another way, the shape of the portion 60 is complementary to the shape of the portion 58 of the pre-fabricated strut 56. A collective shape and volume of the additively manufactured footplate 52 and the pre-fabricated strut 56 together are adapted to fit inside a shoe (not depicted) of the patient. The shape of each portion 58, 60 may be curved, rounded, circular, semi-circular, or any other shape capable of fitting inside the shoe of the patient and still fall within the scope of the present disclosure. In addition, by having the additively manufactured footplate 52 separate from the additively manufactured calf cuff 54, an additive manufacturing time by the 3D printer 16 of the ankle-foot orthosis 50 is less than three hours. This is a significant reduction in time compared to manufacturing times of one to three weeks at a minimum using the conventional manufacturing methods discussed above relative to FIGS. 1A-2, for example.

In addition, the additively manufactured calf cuff 54 also includes at least one hole in a central portion. In this example, the calf cuff 54 includes two holes 61a, 61b disposed in a vertical configuration. Specifically, a first hole 61a is disposed above a second hole 61b. The holes 61a, 61b are adapted to align with holes in a portion of the pre-fabricated strut 56 when the pre-fabricated strut 56 is connected to the additively manufactured calf cuff 54 and the additively manufactured footplate 52, as described more below.

As depicted in FIG. 6, the pre-fabricated strut 56 includes a distal end 62 having an anchor 64 with a center portion 66 having a hole 68, and a pair of fingers 70. Each finger 71 extends from the center portion 66 and also includes a hole 72. The pair of fingers 70 is adapted to extend around an ankle region of the patient. In this example, the pair of fingers 70 outwardly and downwardly extend from the center portion 66 of the anchor 64, positioning the hole 68 in the center portion 66 of the anchor 64 above each hole 72 disposed on each finger 71. So configured, a three-hole configuration on the distal end 62 of the pre-fabricated strut 56 is formed. Alternatively, various other configurations may be used, such as fingers that extend perpendicular to the center portion 66 of the anchor 64, fingers that extend slightly upwardly from the center portion 66 of the anchor 64, and/or various other number of holes included in one or more of the center portion 66 or fingers 71 of the anchor 64 and still fall within the scope of the present disclosure. The anchor 64 reduces the thickness of the ankle region 55, allowing the shape and volume of the additively manufactured footplate 52 to fit inside a shoe of a patient (not shown).

In addition, the pre-fabricated strut 56 also includes a proximal end 74 adapted to be coupled to the additively manufactured calf cuff 54. In this example, the proximal end 74 includes two holes 76 disposed in a vertical orientation. The two holes 76 may comprise a first hole 76a and a second hole 76b, and the first hole 76a may be disposed above the second hole 76b along the same axis as the second hole 76b. Alternatively, more or fewer than two holes 76 may be included in the proximal end 74 and still fall within the scope of the present disclosure.

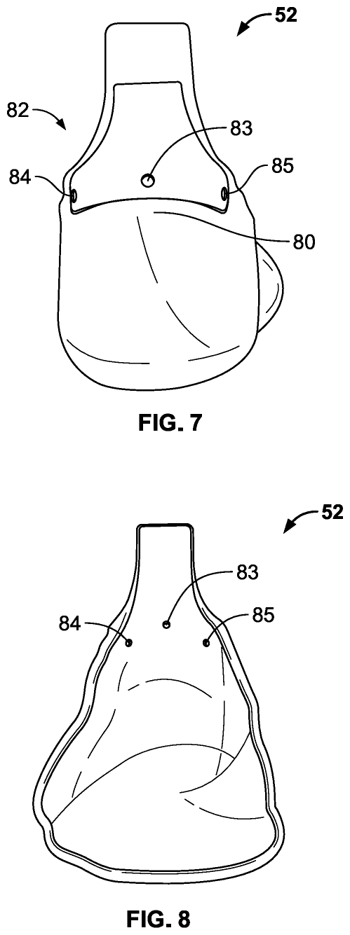

As depicted in FIGS. 7 and 8, the additively manufactured footplate 52 also includes a plurality of holes that align with the holes 68, 72 of the anchor 64 of the pre-fabricated strut 56 when assembled. More specifically, the additively manufactured footplate 52 includes a portion 80 having a plurality of holes 82, such as three holes. A first hole 83 aligns with the hole 68 in the center portion 66 of the distal end 62 of the pre-fabricated strut 56. In addition, a second hole 84 and a third hole 85 align with the holes 72 in the fingers 71 of the distal end 62 of the pre-fabricated strut 56. Once aligned together, a fastening member is inserted through each of the aligned sets of holes of both the additively manufactured footplate 52 and the pre-fabricated strut 56 to secure them together. The fastening member (not depicted) may include any fastening member capable of securing parts together, such as a bolt-screw combination, a rivet, or any other fastener. In addition, an outside portion of the additively manufactured footplate 52 depicted in FIG. 7 includes a standard shape matching that of the pre-fabricated strut 56. Further, an inside portion of the additively manufactured footplate 52 depicted in FIG. 8 includes a custom shape matching that of a patient-user, as understood from the foregoing description and further details below.

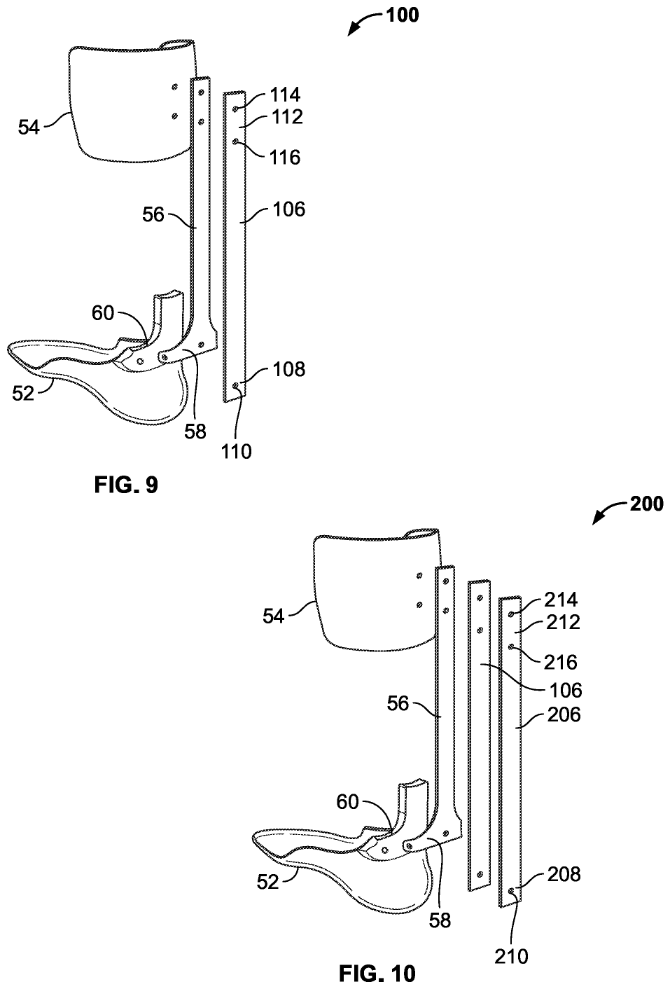

Referring now to FIG. 9, another exemplary ankle-foot orthosis 100 according to another aspect of the present disclosure is depicted. In this example, the ankle-foot orthosis 100 includes the same additively manufactured footplate 52, additively manufactured calf cuff 54, and pre-fabricated strut 56 as the exemplary ankle-foot orthosis 50 of FIGS. 4-8. Unlike the ankle-foot orthosis 50, however, the ankle-foot orthosis 100 includes a plate 106 coupled to the pre-fabricated strut 56, as explained below. Parts of the ankle-foot orthosis 100 that are the same as parts of the ankle-foot orthosis 50 have the same reference numbers as the ankle-foot orthosis 50 and are not explained here again for the sake of brevity.

More specifically, the ankle-foot orthosis 100 includes the plate 106 connected to each of the pre-fabricated strut 56, the additively manufactured calf cuff 54, and the additively manufactured footplate 52 to help increase flexural stiffness of the ankle-foot orthosis 100, for example. In one example, the plate 106 includes a distal portion 108 having a single hole 110. The single hole 110 aligns with the hole 68 of the distal end 62 of the pre-fabricated strut 56 and the hole 83 in the additively manufactured footplate 52 when all are assembled. The plate 106 further includes a proximal end 112 that includes two holes 114, 116 also disposed in a vertical configuration matching that of both the pre-fabricated strut 56 and the additively manufactured calf cuff 54. So configured, the holes 114, 116 of the plate 106 align with the holes 76a, 76b of the pre-fabricated strut 56 and the holes 61a, 61b (FIG. 5) of the additively manufactured calf cuff 54.

Referring now to FIG. 10, another exemplary ankle-foot orthosis 200 according to another aspect of the present disclosure is depicted. In this example, the ankle-foot orthosis 200 includes the same additively manufactured footplate 52, additively manufactured calf cuff 54, and pre-fabricated strut 56 as the exemplary ankle-foot orthosis 50 of FIGS. 4-8. Unlike the ankle-foot orthosis 50, however, the ankle-foot orthosis 200 includes two additional plates coupled to the pre-fabricated strut 56, as explained more below. Parts of the ankle-foot orthosis 200 that are the same as parts of the ankle-foot orthosis 50 have the same reference numbers as the ankle-foot orthosis 50 and are not explained here again for the sake of brevity.

More specifically, the ankle-foot orthosis 200 includes the plate 106 of the ankle-foot orthosis 100 of FIG. 9 and another plate 206 connected to each of the plate 106, the pre-fabricated strut 56, the additively manufactured calf cuff 54 and the additively manufactured footplate 52 to again help further increase flexural stiffness of the ankle-foot orthosis 200, for example. In one example, the plate 206 includes a distal portion 208 having a single hole 210. The single hole 210 aligns with the hole 110 of the plate 106, the hole 68 of the distal end 62 of the pre-fabricated strut 56, and the hole 83 in the additively manufactured footplate 52 when all are assembled. The plate 206 further includes a proximal end 212 that includes two holes 214, 216 also disposed in a vertical configuration matching each of the plate 106, the pre-fabricated strut 56 and the additively manufactured calf cuff 54. So configured, the holes 214, 216 of the plate 206 align with the holes 76a, 76b of the pre-fabricated strut 56, the holes 114, 116 of the plate 106, and the holes 61a, 61b of the additively manufactured calf cuff 54.

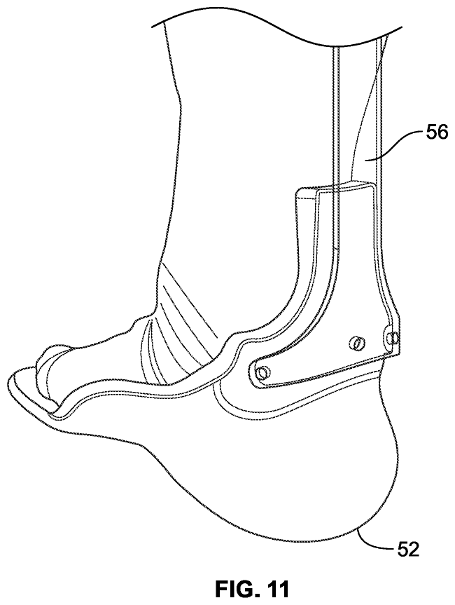

Referring now to FIG. 11, the additively manufactured footplate 52 and a portion of the pre-fabricated strut 56 are depicted with a portion of the patient's foot, ankle and calf disposed therein. As shown, the patient's foot, the back portion of the heel, and the lower portion of the calf are in direct contact with an inner surface of the additively manufactured footplate 52. The additively manufactured footplate 52 exactly matches the shape and size of the patient's foot and calf due to the additively manufacturing process of the system 10 described above, for example.

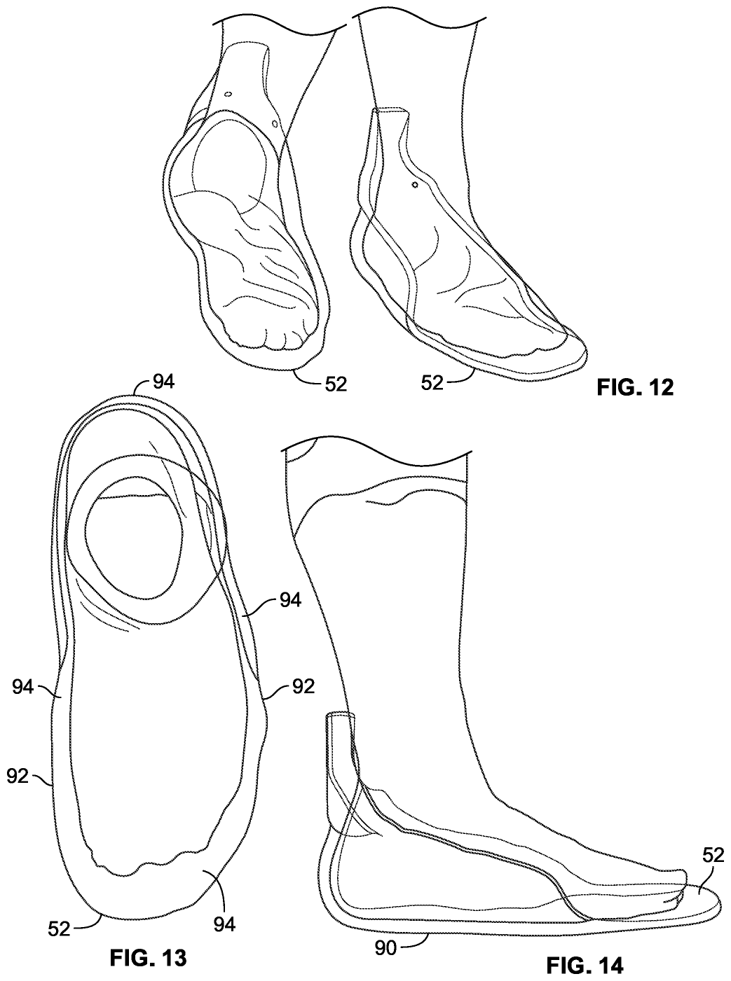

This is further illustrated in FIGS. 12-14. More specifically, FIGS. 12-14 depict an exemplary additively manufactured footplate 52 having an exemplary patient's foot, ankle and a portion of the calf disposed therein. The additively manufactured footplate 52 exactly fits the dimensions of the patient's foot, ankle, and a portion of the calf. The footplate-is flexible, dynamically moving with the movement of portions of the foot, ankle, and calf, as depicted therein. As depicted in FIG. 13, the patient's foot contacts a bottom portion 90 of the additively manufactured footplate 52, but does not directly contact side portions 92, leaving a space 94 between an outer edge of the additively manufactured footplate 52 and the patient's foot. This provides more room for movement of the patient's foot within the additively manufactured footplate 52, increasing comfort for the patient, for example.

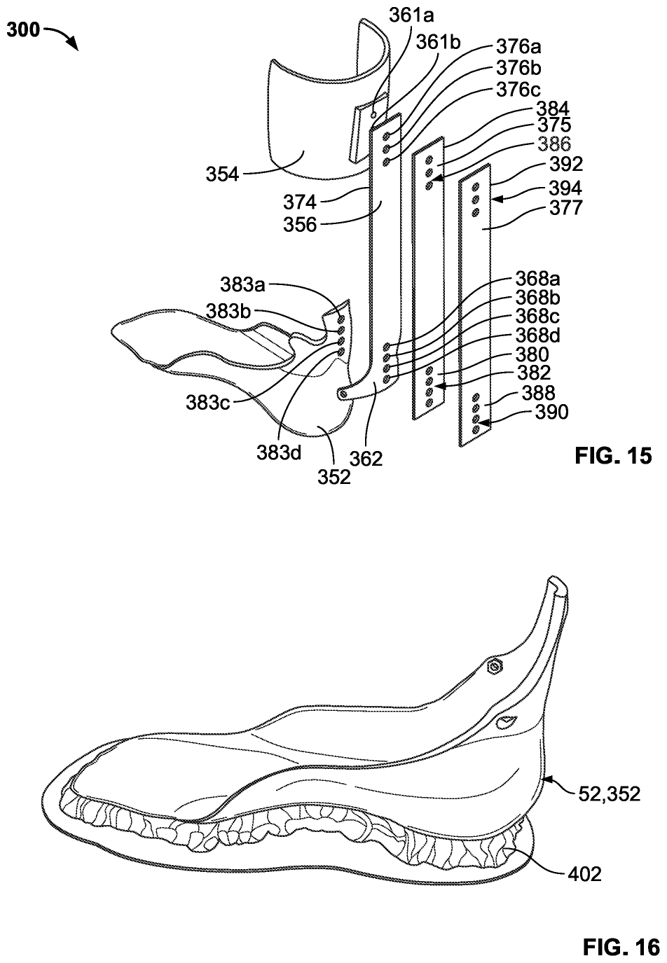

Referring now to FIG. 15, another exemplary ankle-foot orthosis 300 according to another aspect of the present disclosure is depicted. In this example, the ankle-foot orthosis 300 includes an additively manufactured footplate 352, an additively manufactured calf cuff 354, and a pre-fabricated strut 356 similar to the exemplary ankle-foot orthosis 50 of FIGS. 4-8 and the ankle-foot orthosis 200 of FIG. 10. However, unlike the ankle-foot orthoses 50 and 200, the additively manufactured footplate 352, the additively manufactured calf cuff 354 and the pre-fabricated strut 356 each include more holes, as explained more below. Parts of the ankle-foot orthosis 300 that are the same as parts of the ankle-foot orthosis 50 are numbered 300 more than the ankle-foot orthosis 50. Those parts are not explained here again for the sake of brevity.

In particular, the additively manufactured footplate 352 includes four holes 383a, 383b, 383c, and 383d (instead of a single hole) disposed in a vertical configuration along a center portion of the footplate 352. Similarly, a distal end 362 of the pre-fabricated strut 356 includes four holes 368a, 368b, 368c, and 368d also disposed in a vertical configuration on a center portion (instead of a single hole) that align with the four holes 383a, 383b, 383c and 383d of the additively manufactured footplate 352 when assembled. In addition, a center portion of the additively manufactured calf cuff 354 includes three holes 361a, 361b, 361c (instead of two holes) disposed in a vertical configuration. In a similar manner, a center portion of a proximal end 374 of the pre-fabricated strut 356 includes three holes 376a, 376b, and 376c (instead of two holes) disposed in a vertical configuration that align with the holes 361a, 361b, and 361c in the additively manufactured calf cuff 354 when assembled.

Like the ankle-foot orthosis 200, the ankle-foot orthosis 300 may also include one or two additional plates 375, 377 connected to the pre-fabricated strut 356, the additively manufactured calf cuff 354 and the additively manufactured footplate 352 to again help further increase flexural stiffness of the ankle-foot orthosis 300, for example. In this example, the plate 375 includes a distal end 380 with four holes 382 and a proximal end 384 with three holes 386. The four holes 382 in the distal end 380 align with the four holes 368a-368d in the distal end 362 of the pre-fabricated strut 356, and the three holes 386 in the proximal end 384 align with the three holes 376a-376c of the pre-fabricated strut 356. A second plate 377 may also be secured to the first plate 375 and includes the same features of the first plate 375. In particular, the second plate 377 includes a distal end 388 with four holes 390 that align with the four holes 382 in the first plate 375 when connected to the first plate 375. In addition, the second plate 377 also includes a proximal end 392 with three holes 394 that align with the holes 386 in the proximal end 384 of the first plate 375 when connected to the first plate 375. As will be appreciated, more or fewer holes may be included in each of the first and second plates 375, 377, the pre-fabricated strut 356, and the additively manufactured footplate 352, for example, and still fall within the scope of the present disclosure.

In addition, as will be appreciated, the pre-fabricated strut 356, like all of the other pre-fabricated struts discussed above, does not need to be straight and may include various different cross-sectional shapes used to increase flexural stiffness, for example. This is particularly useful when additional plates are used. As an example, the pre-fabricated strut 356 may include a C-, V-, or I-shaped cross-section to increase the bending stiffness or may alternatively include a corrugated structure and still fall within the scope of the present disclosure.

Referring now to FIG. 16, the additively manufactured footplate 52, 352 of the present disclosure is depicted with a tree structure support 402 used in a material extrusion process of the additive manufacturing method of the present disclosure. In particular, the additively manufactured footplate 52, 352 is adapted to be formed by the tree structure support 402 during the additive manufacturing process. In this example, the additively manufactured footplate 52, 352, and the tree structure support 402 include a single material, such as Nylon 12 material. The tree support structure 402 acts as a support structure for material extrusion for the additively manufactured footplate 52, 352, and eliminates the use of support material in the MEX process, as explained more below.

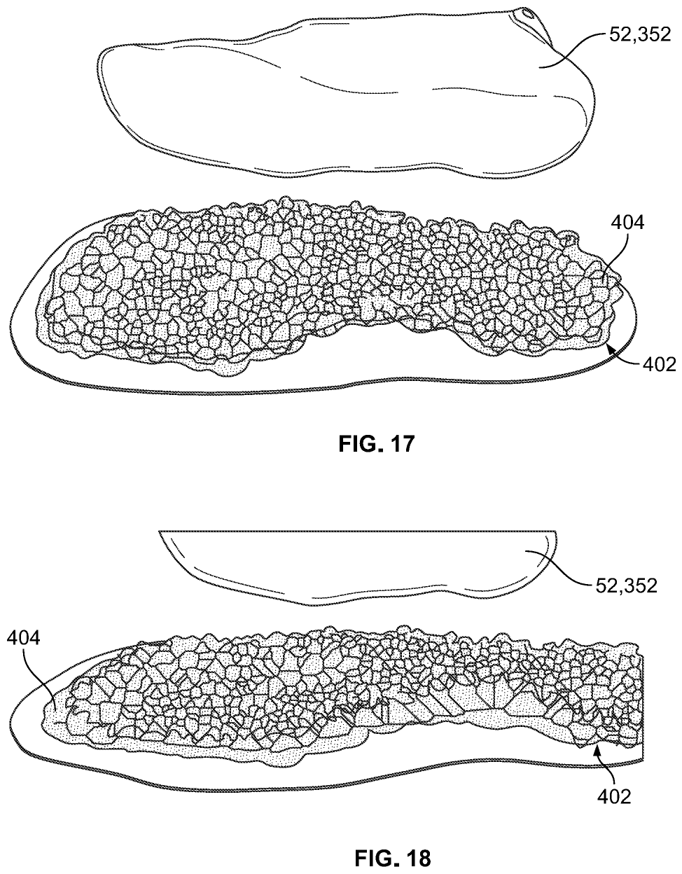

Referring now to FIGS. 17 and 18, the additively manufactured footplate 52, 352 is depicted after separation of the additively manufactured footplate 52, 352 from the tree support structure 402. As shown therein, the tree support structure 402 includes at least one anchor 404 and in some examples a plurality of anchors. The at least one anchor 404 or the plurality of anchors are removable from the additively manufactured footplate 52, 352 without using a chemical for dissolving a support material. In addition, the surface roughness of the footplate 52, 352 is still good after separation of the footplate 52, 352 from the tree support structure 404. As a result, an aggressive abrasive process for polishing and potentially damaging the strength of the additively manufactured footplate 52, 352 is avoided.

There are several advantages to eliminating the use of any support material in the additive manufacturing method of the present disclosure. For example, the MEX speed is faster without printing any extra material that will be removed, and the time to purge a nozzle of build and support materials in each layer is also saved, resulting in a more efficient additive manufacturing process. In addition, the time for chemical dissolution of any support material is also eliminated and the high-temperature vapor can also be avoided in the clinical operation. Further, the potential of a residual chemical agent used to dissolve support material on one or more of the additively manufactured, e.g., 3D printed, footplate 52, 352 or calf cuff 54, 354 of the ankle-foot orthosis 50, 300 is eliminated. Because the residual chemical agent is eliminated, the patient does not have any prolonged contact with the chemical agent disposed on the ankle-foot orthosis during use of the ankle-foot orthosis, and the safety of the patient is ensured. In addition, the manufacturing time needed is also reduced, which likewise reduces the costs involved.

In view of the foregoing, it will be appreciated that the ankle-foot orthoses 50, 100, 200, 300 of the system 10 of the present disclosure may be additively manufactured according to the following exemplary method. In particular, and in one example, a method of additively manufacturing the ankle-foot orthosis 50, 100, 200, 300 comprises providing the tree structure support 402 as a support structure for material extrusion of the ankle-foot orthosis 50, 100, 200, 300. The method further includes fabricating the tree structure support 404 and one or more of the footplate 52, 352, and the calf cuff 54, 354 with a single material. The method still further includes eliminating any support material used in the material extrusion process and removing the tree structure support 402 from one or more of the footplate 52, 352, and the calf cuff 54, 354 without using any chemical for dissolving any support material.

In some examples, fabricating the tree structure support 404 and one or more of the footplate 52, 352 and the calf cuff 54, 354 with a single material may comprise fabricating the tree structure support 402 and one or more of the footplate 52, 352 and the calf cuff 54, 354 with Nylon 12 material. In addition, removing the tree structure support 402 from one or more of the footplate 52, 352 and the calf cuff 54, 354 without using any chemical for dissolving any support material comprises removing at least one anchor 404 of the tree structure support 402 from one or more of the footplate 52, 352 and the calf cuff 54, 354 without using any chemical for dissolving a support material.

In view of the foregoing, and in addition to the many advantages described above, the system 10 and method for additively manufacturing the ankle-foot orthosis 50, 100, 200, 300, and the ankle-foot orthosis 50, 100, 200, 300 includes at least the following advantages. For example, the system 10 of FIGS. 3A and 3B can reduce manufacturing time, decrease the artisan-based variability, record the 3D digital shapes, and enable a one day visit to the patient center 12. As a result, the diagnosis, design, manufacturing, and evaluation of the ankle-foot orthosis 50, 100, 200, 300 can be completed in a same day visit to the patient center 12, such as a patient clinic.

Different from conventional AFO manufacturing, the 3D scanning and digitalization design of the system 10 and method of the present disclosure solve common issues, such as non-standardized customization. Further, the system 10 and the ankle-foot orthosis 50, 100, 200, and 300 of the present disclosure are capable of 3D digital record tracking of every patient, wherein the shape of the limb and symptoms in every step of the rehabilitation plays an important role for the evolution of the patient. In addition, the 3D-printed 3-part ankle-foot orthosis 50, 100, 200, 300 is able to track patient records, such as the shape of the patient before and after the ankle-foot orthosis elaboration, as well as motion during the process of rehabilitation, using the inertial measurement unit (IMU) 59, for example. The typical material fatigue and impact strength between two adjacent layers in a layer-by-layer process are strengthened due to the tree support structure 402 and detrimental side effects of using chemical agents to remove old support material (which the tree support structure replaces), such as rashes on patients' skin, disposal fee for use of high-temperature washer needed when using the chemical agents, are eliminated.

Further, by pre-bending the prefabricated strut 56, 356 loading from walking is distributed to the additively manufactured footplate 52, 352, thereby eliminating a need for thick support in an ankle area of the footplate, as required in conventional designs. Moreover, the flexural bending of the pre-fabricated strut 56, 356 determines a dynamic response of the ankle-foot orthosis 50, 100, 200, 300, and is also important for comfort and functionality. Still further, the ankle-foot orthosis 50, 100, 200 300 of the present disclosure is slim and comfortable for users and adapts well to the morphology of the patient's foot, the top of a tennis shoe, and is not clumsy, compared to conventional non-customized ankle-foot orthoses in the market.

The following additional considerations apply to the foregoing discussion. Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components for example configurations may be implemented as a combined structure or component. Similarly, structures and functionality presented as a single component may be implemented as separate components. These and other variations, modifications, additions, and improvements fall within the scope of the subject matter herein.

As used herein any reference to “one implementation,” “one embodiment,” “one example,” “an implementation,” “an embodiment,” or “an example” means that a particular element, feature, structure, or characteristic described in connection with the implementation is included in at least one implementation. The appearances of the phrase “in one implementation” or “in one embodiment” or “in one example” in various places in the specification are not necessarily all referring to the same implementation.

Some implementations may be described using the expression “coupled” along with its derivatives. For example, some implementations may be described using the term “coupled” to indicate that two or more elements are in direct physical or electrical contact. The term “coupled,” however, may also mean that two or more elements are not in direct contact with each other, but yet still co-operate or interact with each other. The implementations are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

In addition, use of the “a” or “an” are employed to describe elements and components of the implementations herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for the system and method disclosed herein. Thus, while particular implementations and applications have been illustrated and described, it is to be understood that the disclosed implementations are not limited to the precise construction and components disclosed herein. Various modifications, changes, and variations, which will be apparent to those skilled in the art, may be made in the arrangement, operation, and details of the method and apparatus disclosed herein without departing from the spirit and scope defined in the appended claims.

1. An ankle-foot orthosis comprising:
an additively manufactured footplate;
a calf cuff separate from the additively manufactured footplate; and
a pre-fabricated strut connecting the additively manufactured footplate to the calf cuff, the pre-fabricated strut having a thickness and a width adapted to define a patient-specific stiffness about an ankle joint of a patient.

2. The ankle-foot orthosis of claim 1, wherein the calf-cuff is additively manufactured, and a separation of the additively manufactured footplate and the additively manufactured calf cuff allows an additive manufacturing time of the ankle-foot orthosis to be reduced.

3. The ankle-foot orthosis of claim 1, the pre-fabricated strut including a distal end having an anchor with a center portion having a hole, and a pair of fingers, each finger extending from the center portion and including a hole, the pair of fingers adapted to extend around an ankle region of the patient.

4. The ankle-foot orthosis of claim 3, wherein the pair of fingers outwardly and downwardly extend from the center portion of the anchor, positioning the hole in the center portion of the anchor above each hole disposed on the finger, forming a three-hole configuration on the distal end of the pre-fabricated strut.

5. The ankle-foot orthosis of claim 1, the pre-fabricated strut including one or more of a proximal end adapted to be coupled to the additively manufactured calf cuff, the proximal end having two holes, or a cross-section having a C-, V-, or I-shape.

6. The ankle-foot orthosis of claim 5, wherein the two holes of the proximal end of the pre-fabricated strut are disposed in a vertical orientation and comprise a first hole and a second hole, the first hole disposed above the second hole along the same axis as the second hole.

7. The ankle-foot orthosis of claim 1, wherein the pre-fabricated strut comprises metal, such as spring steel.

8. The ankle-foot orthosis of claim 1, further comprising one or more plates connected to the pre-fabricated strut to help increase flexural stiffness of the ankle-foot orthosis, each of the plates includes a proximal end having at least two holes adapted to align with the proximal end of the pre-fabricated strut and a distal end having at least one hole adapted to align with the hole in the anchor of the pre-fabricated strut.

9. The ankle-foot orthosis of claim 1, further comprising an inertia measurement unit disposed on the pre-fabricated strut near the additively manufactured calf cuff, the inertia measurement unit adapted to measure one or more of gait motion and patient data during use.

10. The ankle-foot orthosis of claim 9, the inertia measurement unit comprising a processor, memory, a transmitter, and a receiver, such that the receiver receives measured data, the processor processes the data according to at least one module stored on the memory, and the transmitter transmits data to a data station in real-time via a wireless network, the data station using the data to estimate one or more step height, step length, and step duration of the patient.

11. The ankle-foot orthosis of claim 10, wherein the data station one or more of saves, maintains and tracks data relative to records of the patient, including one or more of the shape of the patient before and after use of the ankle-foot orthosis, and motion during the process of patient rehabilitation.

12. An ankle-foot orthosis comprising:
an additively manufactured footplate having an ankle region;
a calf cuff separate from the additively manufactured footplate; and
a pre-fabricated strut connecting the additively manufactured footplate to the calf cuff,
wherein the pre-fabricated strut includes a distal end having an anchor with two fingers forming a two-finger expansion, the anchor having a center portion with a hole and each finger including a hole, the anchor adapted to uniformly spread a load in the ankle region of the additively manufactured footplate, the load below a fracture strength of a material of the additively manufactured footplate, and
wherein the anchor reduces the thickness of the ankle region, allowing a shape and a volume of the additively manufactured footplate to fit inside a shoe of a patient.

13. The ankle-foot orthosis of claim 12, further comprising an ankle bending stiffness and a dynamic response, wherein one or more of the ankle bending stiffness and the dynamic response is adjusted by adding or removing one or more plates to the pre-fabricated strut.

14. The ankle-foot orthosis of claim 12, wherein the additively manufactured footplate is adapted to be formed by a tree structure support during an additive manufacturing process, the additively manufactured footplate and the tree support structure comprising a single material, and wherein the tree support structure includes a support structure for material extrusion for the additively manufactured footplate, and wherein at least one anchor of the tree structure support is removable from the additively manufactured footplate without using a chemical for dissolving a support material.

15. The ankle-foot orthosis of claim 12, the pre-fabricated strut further comprising a proximal end adapted to be coupled to the additively manufactured calf cuff, the proximal end having two holes.

16. The ankle-foot orthosis of claim 15, further comprising one or more plates, each plate having a proximal end and a distal end, the proximal end having two holes adapted to align with the holes of the proximal end of the pre-fabricated strut, and the distal end having a hole adapted to align with the hole in the center portion of the anchor of the distal end of the pre-fabricated strut.

17. The ankle-foot orthosis of claim 12, further comprising an inertia measurement unit disposed on the pre-fabricated strut near the additively manufactured calf cuff, the inertia measurement unit adapted to measure a gait motion of the patient.

18. A method of additively manufacturing an ankle-foot orthosis, the method comprising:
providing a tree structure support as a support structure for material extrusion of the ankle-foot orthosis;
fabricating the tree structure support and one or more of a footplate and a calf cuff with a single material;
eliminating support material used in the material extrusion; and
removing the tree structure support from one or more of the footplate and the calf cuff without using any chemical for dissolving any support material.

19. The method of claim 18, wherein fabricating the at least one tree structure support and one or more of a footplate and a calf cuff with a single material comprises fabricating the at least one tree structure support and one or more of a footplate and a calf cuff with a nylon material.

20. The method of claim 18, wherein removing the tree structure support from one or more of the footplate and the calf cuff without using any chemical for dissolving any support material comprises removing at least one anchor of the tree structure support from one or more of the footplate and the calf cuff without using any chemical for dissolving a support material.

21. A system for additively manufacturing an ankle-foot orthosis, the system comprising:
a communication network;
a scanning device communicatively coupled to the communication network, the scanning device including a memory and at least one processor, the at least one processor of the scanning device executing a scanning module stored on the memory of the scanning device to create a patient-specific scan;
a 3D printer communicatively coupled to the communication network;
a cyber design system having a computing device communicatively coupled to the communication network, the scanning device, and the 3D printer, the computing device of the cyber design system having a memory, at least one processor, a transmitter, and a receiver, the computing device receiving data from the scanning device relating to the patient-specific scan; and
a module stored in the memory of the computing device of the cyber design system and executable by the at least one processor of the computing device of the cyber design system to:
(1) receive data from the scanning device relating to the patient-specific scan; and
(2) create a patient-specific ankle-foot orthosis profile based at least in part on the data received from the scanning device, the patient-specific ankle-foot orthosis profile adapted to be transmitted to the 3D printer to implement the patient-specific ankle-foot orthosis profile;
wherein the scanning device creates the patient-specific scan and the 3D printer receives the patient-specific ankle-foot orthosis profile from the computing device of the cyber design center and implements the patient-specific ankle-foot orthosis profile to additively manufacture a footplate of an ankle-foot orthosis and a calf cuff of the ankle-foot orthosis, the calf cuff separate from the footplate, within one day.

22. The system of claim 21, the scanning device further comprising a transmitter and a receiver, the transmitter transmitting the scanning data to the computing device of the cyber design center.

23. The system of claim 21, the 3D printer having a memory, at least one processor, a transmitter and a receiver, the receiver of the 3D printer receiving the patient-specific ankle-foot orthosis profile from the computing device of the cyber design center and the at least one processor of the 3D printer executing the patient-specific ankle-foot orthosis profile to additively manufacture the ankle-foot orthosis within one day.

24. An ankle-foot orthosis comprising:
an additively manufactured footplate;
a calf cuff separate from the additively manufactured footplate; and
a pre-fabricated strut connecting the additively manufactured footplate to the calf cuff, the pre-fabricated strut having a thickness and width adapted to define a patient-specific stiffness about an ankle joint of a patient,
wherein the stiffness of the pre-fabricated strut is suited to one or more gait needs or requirements of each patient.

25. An ankle-foot orthosis comprising:
an additively manufactured footplate;
a calf cuff separate from the additively manufactured footplate; and
a pre-fabricated strut connecting the additively manufactured footplate to the calf cuff, the pre-fabricated strut having a thickness and width adapted to define a patient-specific stiffness about an ankle joint of a patient, and a portion with a shape;
wherein the additively manufactured footplate has a portion with a shape complementary to the shape of the portion of the pre-fabricated strut.

26. An ankle-foot orthosis comprising:
an additively manufactured footplate;
a calf cuff separate from the additively manufactured footplate; and
a pre-fabricated strut connecting the additively manufactured footplate to the calf cuff, the pre-fabricated strut having a thickness and width adapted to define a patient-specific stiffness about an ankle joint of a patient,
wherein a collective shape and volume of the additively manufactured footplate and the pre-fabricated strut together are adapted to fit inside a shoe of the patient.

27. The ankle-foot orthosis of claim 1, wherein the stiffness of the pre-fabricated strut is suited to one or more gait needs or requirements of each patient.

28. The ankle-foot orthosis of claim 1, wherein the pre-fabricated strut includes a portion with a shape, and wherein the additively manufactured footplate has a portion with a shape complementary to the shape of the portion of the pre-fabricated strut.

29. The ankle-foot orthosis of claim 1, wherein a collective shape and volume of the additively manufactured footplate and the pre-fabricated strut together are adapted to fit inside a shoe of the patient.
